(19) United States
(12) Patent Application Publication
(10) Pub. No.: US 2026/0067553 A1
(43) Pub. Date: Mar. 5, 2026

(54) IMAGING ASSEMBLY WITH MULTIPLE DEGREES OF MOVEMENT

(71) Applicant: Med Smart Hub Ltd., Caesarea Industrial Park (IL)

(72) Inventors: Dean Cohen, Carmiel (IL); Kfir Solomon, Zoran (IL); Sefi Shachrur, Pardes-Hana Karkur (IL); Gil Bachar, Tel Aviv (IL)

(73) Assignee: Med Smart Hub Ltd., Caesarea Industrial Park (IL)

(21) Appl. No.: 18/822,800

(22) Filed: Sep. 3, 2024

(51) Int. Cl. H04N 23/57; A61B 1/045; A61B 1/05; H04N 23/50; H04N 23/52; H04N 23/695

(52) U.S. Cl. CPC H04N 23/57; A61B 1/045; A61B 1/05; H04N 23/555; H04N 23/695; H04N 23/52

(57) ABSTRACT

An imaging assembly includes an external hollow member, which extends from a housing, an internal member disposed in the external hollow member, an imaging probe pivotally coupled to the internal member, and an actuator mechanism coupled to the external hollow member and to the internal member configured to move the external hollow member and the internal member in independent degrees of movement.

### FIELD OF THE INVENTION

The present invention relates generally to minimally invasive surgery, and particularly to an imaging assembly, which is capable of being moved in multiple degrees of freedom to provide improved imaging.

### BACKGROUND OF THE INVENTION

One of the first steps during a laparoscopic surgical procedure involves insufflation of the abdomen with nitrogen or carbon dioxide gas. The resulting expansion of the abdomen reduces the risk of injury to the contents of the abdomen during subsequent insertion of the ports and also allows the surgeons more freedom and space to manipulate instruments and perform the surgery.

Laparoscopic surgery is generally performed with a laparoscope comprising a camera and a telescope lens system inserted into the abdomen in one small incision. In order to minimize risk of injury to the patient, it is preferable to observe the exit ports of all cannulas every time an instrument is inserted or withdrawn. Such observation currently requires that the tip of the laparoscope be directed toward a particular port. This would then result in the loss of visualization of the surgical field, which interrupts the surgical procedure and interrupts the use of the surgical instruments until the surgical field can again be visualized with the laparoscope.

PCT Patent Application WO 2022/074661 describes an improved imaging assembly, which has an actuator that can tilt an imaging shaft of the imaging assembly about a joint, so that the shaft rotates about one or more rotation axes

### SUMMARY OF THE INVENTION

The present invention seeks to provide an imaging assembly, which is capable of being moved in multiple degrees of freedom to provide improved imaging, as is described more in detail hereinbelow.

There is thus provided in accordance with a non-limiting embodiment of the present invention an imaging assembly including an external hollow member, which extends from a housing, an internal member disposed in the external hollow member, an imaging probe pivotally coupled to the internal member, and an actuator mechanism coupled to the external hollow member and to the internal member configured to move the external hollow member and the internal member in independent degrees of movement.

The invention provides advantages over the prior art, such as but not limited to, providing omnidirectional vision without interference to the surgeon, which is achieved by the relatively small dimensions of the device and actuation of the probe located at the distal end of the device. Another advantage is the small diameter of the entrance cannula, which is achieved by putting the motors outside of the abdomen. Another advantage is preventing haze on the optic and viewing elements. Another advantage is the unique method of mounting the device on a jig base.

### BRIEF DESCRIPTION OF THE DRAWINGS

The present invention will be understood and appreciated more fully from the following detailed description, taken in conjunction with the drawing in which:

FIG. 1 is a simplified illustration of an imaging assembly, constructed and operative in accordance with a non-limiting embodiment of the present invention.

FIG. 2 is a simplified illustration of the internal mechanism of the imaging assembly, in accordance with a non-limiting embodiment of the present invention.

FIG. 3 is a simplified illustration of gas flow through the imaging assembly, in accordance with a non-limiting embodiment of the present invention.

FIG. 4 is a simplified illustration of a reference jig which has a base for coupling thereto the housing of the imaging assembly, in accordance with a non-limiting embodiment of the present invention.

FIG. 5 is a simplified illustration of a sharp instrument passing through an operational port of the base of the reference jig to make an incision.

FIG. 6 is a simplified illustration of an external hollow member of the imaging assembly passing through the operational port into a cannula of the reference jig.

FIG. 7 is a simplified illustration of the imaging assembly fully coupled to the reference jig.

### DETAILED DESCRIPTION OF EMBODIMENTS

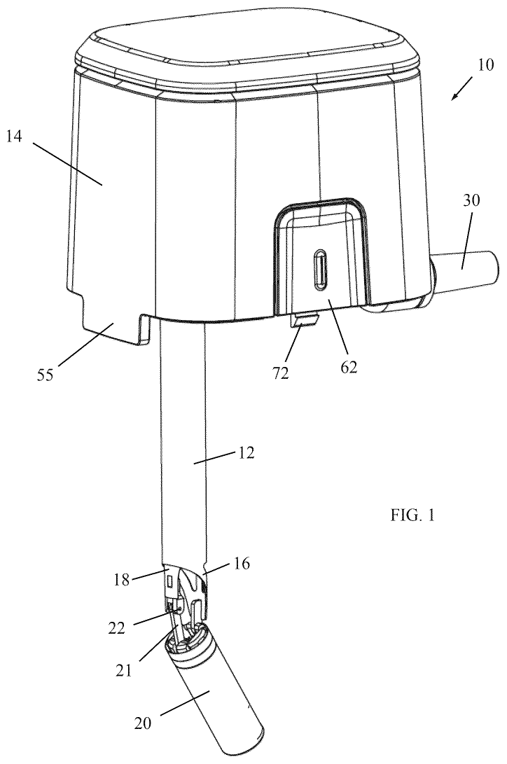

Reference is now made to FIG. 1, which illustrates an imaging assembly 10, constructed and operative in accordance with a non-limiting embodiment of the present invention.

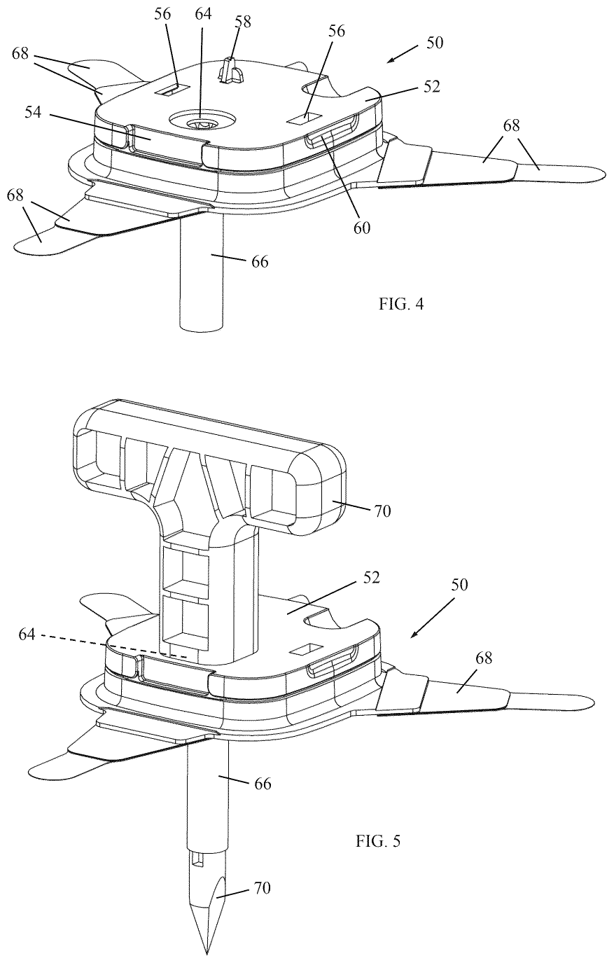

The imaging assembly 10 includes an external hollow member 12, such as a rod or shaft 12, which extends from a housing 14. The external hollow member 12 may extend perpendicularly from housing 14, or at other non-perpendicular angles. The external hollow member 12 may include a distal portion 16, which may be a pointed cutting blade, in which case external hollow member 12 serves as a trocar which may be used to puncture skin. Alternatively, distal portion 16 may be blunt, in which case external hollow member 12 may enter through a separately made incision, as described below with reference to FIGS. 4-7.

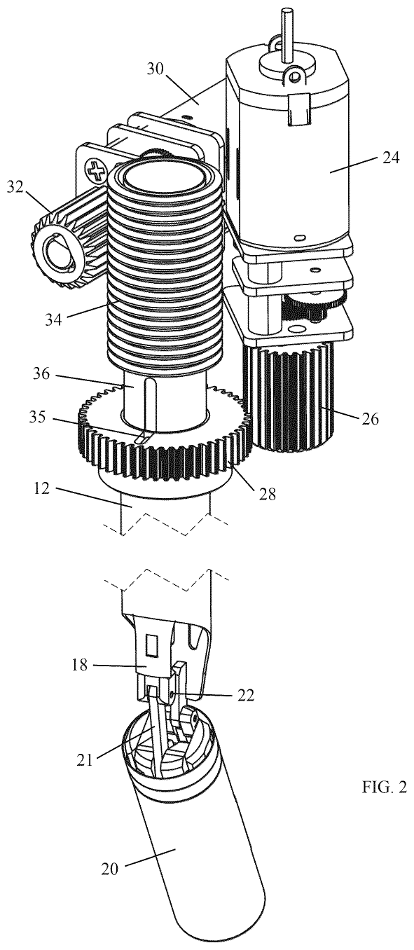

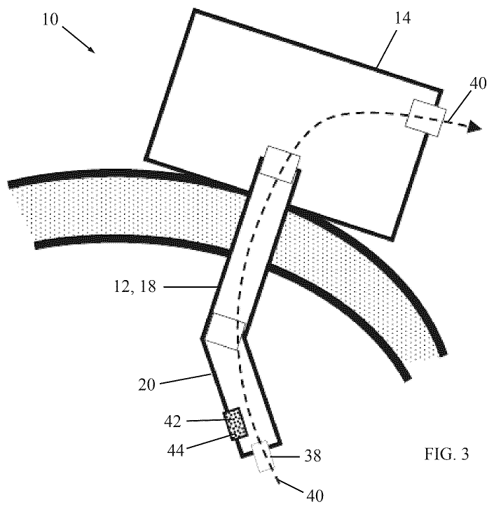

An internal member 18 is disposed in external hollow member 12. Internal member 18 may be hollow. Internal member 18 is preferably, but not necessarily, concentric with the external hollow member 12. An imaging probe 20 may be pivotally coupled by linkage arms 21 to a hinge 22 (such as a hinge pin) located at a distal end of internal member 18. One or more optical elements 42 (not seen in FIG. 2 but seen in FIG. 3) may be disposed on imaging probe 20 and allow the visualization of the abdomen. In addition, other electric components 44 (not seen in FIG. 2 but seen in FIG. 3) may be disposed on imaging probe 20.

Reference is now made to FIG. 2, which illustrates an internal actuator mechanism of the imaging assembly, in accordance with a non-limiting embodiment of the present invention. The actuator mechanism may be mounted inside housing 14 of FIG. 1. The actuation mechanism may be designed such that all actuators are outside the patient's body in an operation. Such a design is advantageous for the reduction of the diameter of external hollow member 12 which reduces the size of the abdomen incision.

The actuator mechanism may include a first actuator 24 (such as a servomotor, a DC motor, a step motor, etc.) which rotates a first spur gear 26. The first spur gear 26 may mesh with a second spur gear 28 which is coupled to external hollow member 12. Thus, rotation of first actuator 24 causes rotation of external hollow member 12 about the longitudinal axis of external hollow member 12. The rotation may be clockwise or counterclockwise. In another embodiment (not illustrated) hollow member may be rotated directly by actuator 24 which is concentric with it. In yet another embodiment (not illustrated) actuation of hollow member 12 by actuator 24 may be transferred by one or more intermediate members, such as but not limited to, a gear or several gears, a timing belt, etc.

A second actuator 30 (such as a servomotor, step motor, etc.) rotates a pinion gear 32, which meshes with a rack 34, which is preferably a cylindrical rack and coaxial with hollow member 12. In one non-limiting embodiment, rack 34 is a non-helical, spur gear. In another non-limiting embodiment, rack 34 is a cylindrical non-worm gear, i.e., all its teeth are parallel to each other and perpendicular to its axis.

The pinion gear 32 meshes with the teeth of rack 34 to form a rack and pinion assembly. The rack 34 has a non-toothed shaft 36 which is coupled to internal member 18. Thus, rotation of second actuator 30 causes rack 34 and internal member 18 to move linearly (up and down in the sense of FIG. 2).

The linear (up and down) motion of internal member 18 creates a linear force (either upwards or downwards) that makes linkage arms 21 rotate about hinge 22. This makes imaging probe 20 rotate either upwards or downwards about hinge 22. The field of view (FOV) of one or more optical elements 42 disposed on imaging probe 20 may shift up\down following the linear motion of internal member 18.

Although rack 34 could be arranged not to rotate, in a preferred embodiment, rack 34 is coupled to second spur gear 28, such as by a pin 35, so that rack 34 and internal member 18 rotate together with external hollow member 12 (which is why rack 34 is cylindrical; if rack 34 does not rotate, then a linear rack could be used). In any case, the linear motion of internal member 18 is independent of rotation of external hollow member 12.

The imaging probe 20 may include an imaging device, such as but not limited to, a camera, ultrasound sensor or other suitable imaging modality sensor. The imaging device may view the internal portion of the patient by means of one or more optical elements 42 (FIG. 3), for example, an illumination source (that can generate light to illuminate the area to be imaged), and light modification elements, such as one or more lenses or filters.

In order to reduce the incision size, external hollow member outer diameter (OD) reduction is important. In some cases, wiring (electrical, optical etc.) passes in the internal volume of internal member 18. External hollow member 12 and internal member 18 may be hollow tubes which have preferably minimal wall thickness (OD minus internal diameter (ID) divided by 2). External hollow member 12 and\or internal member 18 may be made of a low wall thickness metal such as (but not limited to) stainless steel tube, with a wall thickness of 50-200 micrometers (μm).

If external hollow member 12 is used as a trocar to make the incision, by means of distal portion 16 (FIG. 1) being a pointed cutting blade, then second actuator 30 may be used to initially retract imaging probe 20 fully inside of external hollow member 12.

Reference is now made to FIG. 3. The imaging assembly 10 is placed on top of a patient body (e.g., abdomen comprising skin, fat, peritoneum, etc.). Imaging probe 20 may be formed with one or more gas-inlet apertures 38 (of any size and shape). As stated above, many laparoscopic surgical procedures involve insufflation of the abdomen with abdomen inflow gas 40, such as nitrogen or carbon dioxide gas; in a typical surgery, the abdomen is inflated with abdomen inflow of up to 40-50 liter per minute (LPM). FIG. 3 illustrates the motion of the pressurized gas 40 (the pressure is typically 20-100 mbar above atmospheric pressure) through gas-inlet apertures 38. The gas 40 flows past optical elements 42 and/or electronic components 44 (e.g., part of control circuitry and/or temperature sensor, such as a thermistor, illumination source such an LED etc.), and eventually exits to the external world (where the pressure equals atmospheric pressure). In a non-limiting embodiment, gas flow 40 is designed to flow in 0.5-4 LPM or 1-3 LPM or 3-6 LPM. In a non-limiting embodiment, gas flow 40 is designed to flow in 5%-20% of the inflation rate of the abdomen. The gas flow may maintain the temperature on the camera at a safe temperature that does not cause injury or damage to tissues (e.g., no more than 48° C., which is the maximal allowed by regulations for a long operation, e.g. in ISO 60601). On the other hand, heating camera lens may prevent hazing or fogging on the camera lens. Keeping the lens at a constant but allowable temperature may be controlled by the temperature sensor working with a controller operating in a closed loop control.

Reference is now made to FIGS. 4-7, which illustrate another embodiment of imaging assembly 10, in which the external hollow member 12 is not a trocar with a sharp tip, and instead the external hollow member 12 enters through a separately made incision.

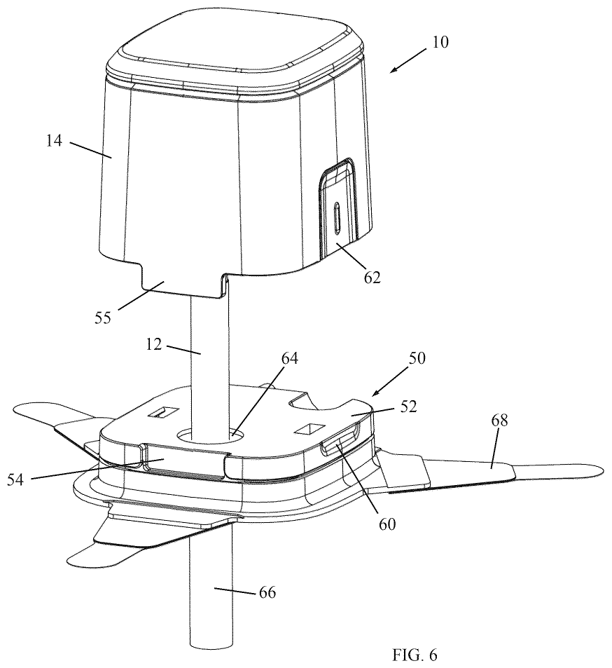

This embodiment may employ a reference jig 50 (FIG. 4), which may include a base 52 for coupling thereto the housing 14 of the imaging assembly 10 (not shown in FIG. 4, but as shown in FIG. 6). Base 52 may be formed with one or more registration features that ensure the housing 14 is properly aligned with reference jig 50. The registration features may include, without limitation, a notch or crevice 54 which receives a tongue or other protrusion 55 (FIGS. 1 and 6) of housing 14, and one or more female alignment elements 56 (such as but not limited to, apertures of any shape, such as non-circular as shown or other shapes) which may receive pins or other features (not shown) that protrude from the underside of housing 14, and/or one or more male alignment elements 58, such as but not limited to, lugs, pins, or other elements that protrude from base 52 and which may enter female features (not shown) that are formed on the underside of housing 14. When assembled together, housing 14 and base 52 may be rigidly coupled to each other so they do not shift one relative to the other by more than 1 mm, without limitation, in any direction.

Base 52 may include a base latch member 60 that couples with a housing latch member 62 (FIGS. 1 and 6) mounted on housing 14. Base 52 may be formed with an operational port or aperture 64, which is aligned with a cannula 66 that extends from the underside of base 52. Base 52 may include one or more mounting members 68, such as but not limited to, tentacles or fingers and the like, that extend outwards on the same plane as the underside of base 52. The use of only two mounting members 68 is less preferred as it may result in a roll motion of the device along a line set between the two mounting members; a third mounting member may stabilize this roll motion. The use of four or more mounting members increases the costs above the minimal necessary. Thus, in the preferred non-limiting illustrated embodiment, there are three mounting members 68. In an embodiment, three mounting members 68 extend symmetrically outwards, for example spaced 120° apart from each other. The mounting members 68 may be adhesive members that bond to the skin of the patient or any other kind of suitable member that can be coupled to the patient.

Reference is now made to FIG. 5. A sharp instrument 70, such as a trocar or puncture needle and the like, may be inserted through operational port 64 and cannula 66 to puncture the patient's skin. After the puncture is made, the surgeon moves cannula 66 through the incision into the patient's body so that base 52 of the reference jig 50 lies against the patient's skin. Base 52 may then be secured to the patient's skin by means of mounting members 68. Base 52 may be rigidly secured to the patient's skin such that it does not rotate by more than 1 degree or 5 degrees, without limitation, around cannula 66. Base 52 may be rigidly secured to the patient's skin such that it does not shift by more than 5 mm, without limitation, relative to the patient's body at any direction. At this point sharp instrument 70 may be removed. In a preferred embodiment, cannula 66 and the tip of sharp instrument 70 may be made such that the gap between them is minimized and the diameter difference at the meeting point is less than few (10-50) micrometers. This design may reduce the risk of body parts or organs (e.g., peritoneum) getting caught in the gap. In a preferred embodiment, cannula 66 is made such that its external face is covered with anti-slip tread to pass through the peritoneum into the body without slipping out of the body from the peritoneum natural motion.

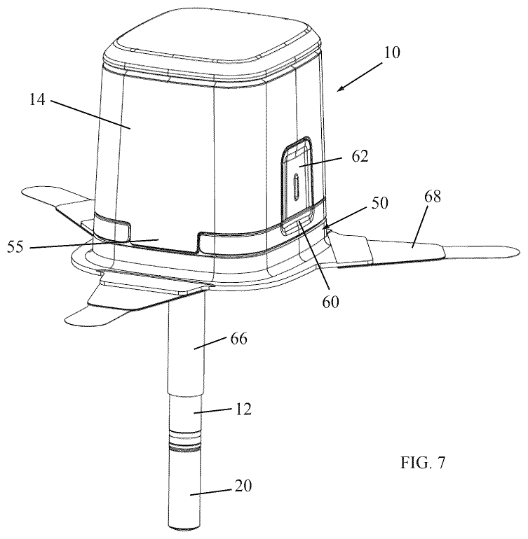

Reference is now made to FIG. 6. In the next step, the external hollow member 12 of the imaging assembly 10 may be passed through operational port 64 into cannula 66. In FIG. 7, the imaging assembly 10 is securely mounted on base 52 of the reference jig 50. The base latch member 60 couples with housing latch member 62 of the housing 14 of the imaging assembly 10. The external hollow member 12 and the imaging probe 20 pass through cannula 66 and are in the operational position to acquire images of the surgical site.

The imaging assembly 10 may include a detection sensor 72 (FIG. 1), which may be mounted at or near housing latch member 62 of the housing 14 or any other portion of housing 14. Detection sensor 72 may be, without limitation, a microswitch, capacitance proximity sensor, magnetic sensor (e.g. a Hall effect sensor coupled to a magnet) and others. If the housing 14 is not attached to the base 52 of the reference jig 50 and if imaging probe 20 is tilted with respect to external hollow member 12, a controller (not shown) in communication with detection sensor 72 causes imaging probe 20 to rotate so that it is aligned straight with external hollow member 12. In this manner, even if the imaging probe had been tilted with respect to external hollow member 12, it is now straight and the imaging assembly 10 can now be easily removed out of cannula 66. For example, when a physician decides to end the surgery and pulls imaging assembly 10 out of the patient's body, detachment of imaging assembly 10 from base 50 is detected by sensor 72. As a response a controller commands probe 20 to rotate to a straight position.

What is claimed is:

1. An imaging assembly comprising:
an external hollow member, which extends from a housing;
an internal member disposed in said external hollow member;
an imaging probe pivotally coupled to said internal member; and
an actuator mechanism coupled to said external hollow member and to said internal member configured to move said external hollow member and said internal member in independent degrees of movement.

2. The imaging assembly according to claim 1, wherein said actuator mechanism comprises a first actuator coupled to said external hollow member, wherein rotation of said first actuator causes rotation of said external hollow member about a longitudinal axis of said external hollow member, and a second actuator coupled to said internal member, wherein rotation of said second actuator causes said internal member to move linearly which causes said imaging probe to rotate with respect to said internal member.

3. The imaging assembly according to claim 2, wherein rotation of said second actuator causes said internal member to move linearly which causes said imaging probe to rotate with respect to said internal member about a second axis perpendicular to said longitudinal axis.

4. The imaging assembly according to claim 1, wherein linear motion of said internal member is independent of rotation of said external hollow member.

5. The imaging assembly according to claim 1, wherein said imaging probe is formed with one or more gas-inlet apertures and said imaging probe comprises at least one optical element arranged with respect to said one or more gas-inlet apertures so that motion of gas flowing through said one or more gas-inlet apertures maintains a temperature of said at least one optical element at a safe temperature that does not cause injury or damage to tissues and prevents hazing or fogging on said at least one optical element.

6. The imaging assembly according to claim 1, further comprising a reference jig comprising a base for coupling thereto said housing, and one or more mounting members for coupling to skin of a patient, wherein said base is formed with an operational port aligned with a cannula that extends from an underside of said base.

7. The imaging assembly according to claim 6, wherein said reference jig comprises one or more registration features that ensure said housing is properly aligned with said reference jig.

8. The imaging assembly according to claim 1, further comprising a detection sensor mounted on a portion of said housing.

9. The imaging assembly according to claim 1, wherein if said housing is not attached to said base, said detection sensor causes said imaging probe to rotate to be aligned straight with respect to said external hollow member.

10. The imaging assembly according to claim 6, wherein said housing is rigidly coupled to said reference jig.

11. The imaging assembly according to claim 1, wherein said external hollow member and internal member are coaxial.

12. The imaging assembly according to claim 1, wherein said internal member is hollow.
